(19) United States
(12) Patent Application Publication
(10) Pub. No.: US 2021/0055432 A1
(43) Pub. Date: Feb. 25, 2021

(54) LIQUID CRYSTAL X-RAY DETECTOR

(71) Applicant: SESIM. LTD, Uiwang-si (KR)

(72) Inventor: Bong Gyu RHO, Suwon-si (KR)

(73) Assignee: SESIM. LTD, Uiwang-si (KR)

(21) Appl. No.: 17/045,752

(22) PCT Filed: Mar. 18, 2019
(86) PCT No.: PCT/KR2019/003105

§ 371 Date: Oct. 6, 2020

(30) Foreign Application Priority Data
Apr. 20, 2018 (KR) 10-2018-0046208

(51) Int. Cl. G01T 1/16; A61B 6/00; H04N 5/32

(52) U.S. Cl. CPC G01T 1/1606; H04N 5/32; A61B 6/4208

(57) ABSTRACT

Disclosed is a liquid crystal X-ray detector capable of obtaining a complete X-ray image of a subject without having to combine smaller partial X-ray images while using an imaging lens much smaller than a liquid crystal layer. The liquid crystal X-ray detector includes a photoconductor unit including a photoconductive layer, a liquid crystal unit provided on the photoconductor unit and including a liquid crystal layer, a read beam output unit outputting a read beam toward the liquid crystal layer, and an imaging lens disposed on a light path in front of the liquid crystal layer. The read beam output unit emits scattered or plane light. The imaging lens has a long axis length that is equal to or smaller than one half of the long axis length of the liquid crystal layer.

### BACKGROUND OF THE INVENTION

### 1. Field of the Invention

The present invention relates to an X-ray detection apparatus. More particularly, the present invention relates to a liquid crystal X-ray detector capable of obtaining an X-ray image of a subject using liquid crystals that change in polarization transmission characteristics with respect to a read beam when being irradiated with X-rays.

### 2. Description of the Related Art

An X-ray imaging apparatus converts a distribution of charges in a layer irradiated with X-rays transmitted through a subject into a digital signal, thereby imaging the interior structure of the subject. The X-ray imaging apparatus is extensively used in medical applications for patient diagnosis, non-destructive building inspection, and the like.

In recent years, X-ray detectors have employed digital technology or liquid crystal cells for improvement in performance thereof. For example, there is an X-ray detector using liquid crystal cells. This X-ray detector is referred to as a liquid crystal X-ray detector or an X-ray sensing liquid crystal detector. The liquid crystal X-ray detector is largely composed of a photoconductive element, a liquid crystal element, a light source, and a photodetector.

In the liquid crystal X-ray detector, when a photoconductive layer of the photoconductive element is irradiated with X-rays and a voltage is applied across two electrodes of the liquid crystal element, the X-rays transmitted through a subject cause polarization in the photoconductive layer while passing through the photoconductive layer. The polarization affects the liquid crystal layer such that the orientations of liquid crystal molecules are changed. Next, a read beam emitted from the light source passes through the liquid crystal layer and the read beam transmitted through the liquid crystal layer is detected by a camera. Through this process, an X-ray image of a subject is obtained.

In such a conventional liquid crystal X-ray detector, the light source that emits the read beam is usually a point light source. The beam emitted from the point light source propagates in a predetermined direction. Therefore, in order to translate the light exiting the liquid crystal layer into an image, an imaging lens larger than the liquid crystal layer is required. In particular, in X-ray chest radiography, an imaging lens having a size of 370 mm×470 mm is typically used. An imaging lens larger than that requires a considerable increase in the cost.

To solve this problem, U.S. Pat. No. 6,052,432 (Patent Document 1) discloses a technology described below. Referring to FIG. 11A, a liquid crystal X-ray detection apparatus disclosed in Patent Document 1 is configured to take an X-ray image while moving a small imaging lens and a camera. Alternatively, as illustrated in FIG. 11B, a plurality of imaging lenses and a plurality of cameras are used to take an X-ray image. That is, with a plurality of smaller imaging lenses than the a liquid crystal layer, an X-ray image having the same size as the liquid crystal layer can be obtained.

However, in the case of using the liquid crystal X-ray detection apparatus disclosed in Patent Document 1, that is, when taking an image while moving a pair of an imaging lens and a camera as illustrated in FIG. 11A, a moving stage that is movable in an X-axis direction and a Y-axis direction is additionally required. In addition, since multiple local X-ray images taken by one camera need to be combined to produce a global X-ray image having the same size as the liquid crystal layer, an image quality is deteriorated at the boundaries between each of the local X-ray images. Therefore, there is a problem that the accuracy and reliability are deteriorated in analyzing and diagnosing a subject using X-ray imaging.

On the other hand, when a liquid crystal X-ray detection apparatus is configured as illustrated in FIG. 11B, since multiple cameras are required, cost is increased. In addition, as with the configuration of FIG. 11A, since multiple local X-ray images are combined to produce a global X-ray image, an image quality is deteriorated at the boundaries between each of the multiple local X-ray images taken by the respective cameras.

### DOCUMENTS OF RELATED ART

### Patent Document

Patent Document 1: U.S. Pat. No. 6,052,432 (issued as of Apr. 18, 2000).

### SUMMARY OF THE INVENTION

An objective of the present invention is to provide a liquid crystal X-ray detector capable of acquiring a seamless X-ray image without using conventional technology in which one camera is moved or multiple cameras are used to take partial images.

Another objective of the present invention is to provide a liquid crystal X-ray detector capable of acquiring a complete X-ray image with an imaging lens much smaller than a liquid crystal layer.

According to one aspect of the present invention, there is provided a liquid crystal X-ray detector including: a photoconductor unit including a photoconductive layer; a liquid crystal unit provided on the photoconductor unit and including a liquid crystal layer; a read beam output unit outputting a read beam toward the liquid crystal layer; and an imaging lens disposed on an optical path in front of the liquid crystal layer.

The read beam output unit may include a light emitting unit that emits scattered light or plane light.

The read beam may include the scattered light or the plane light, and the imaging lens may have a long axis having a length that is shorter than or equal to one-half of the length of a long axis of the liquid crystal layer.

With the liquid crystal X-ray detector according to the present invention, it is possible to acquire a seamless complete X-ray image while using an imaging lens much smaller than a liquid crystal layer.

That is, according to the present invention, a smaller imaging lens than a liquid crystal layer can be used, and a camera moving stage or a plurality of cameras is not required to obtain an X-lay image of a subject. Therefore, the liquid crystal X-ray detector can be manufactured at reduced cost and can improve the accuracy and reliability of an X-ray image formed thereby.

### BRIEF DESCRIPTION OF THE DRAWINGS

The above and other objectives, features, and advantages of the present invention will be more clearly understood from the following detailed description taken in conjunction with the accompanying drawings, in which:

FIG. 1 is a diagram illustrating the overall construction of a liquid crystal X-ray detector according to one embodiment of the present invention;

FIG. 2 is a cross-sectional view of an X-ray sensing liquid crystal panel according to one embodiment of the present invention;

FIG. 3 is a cross-sectional view illustrating a read beam output unit according to a first embodiment of the present invention;

FIG. 4 is a light transmission curve of a photoconductive layer for each wavelength, according to one embodiment of the present invention;

FIG. 5 is a view illustrating a per-wavelength vertical transmittance of a film type polarizing plate widely used in a conventional TFT LCD device;

FIG. 6 is a cross-sectional view illustrating a modification to the first embodiment of the present invention;

FIG. 7 is a perspective view of a light guide plate having a light incident portion, according to one embodiment of the present invention;

FIG. 8 is a cross-sectional view illustrating a read beam output unit according to a second embodiment of the present invention;

FIG. 9 is a cross-sectional view illustrating a read beam output unit according to a third embodiment of the present invention;

FIG. 10 is a cross-sectional view of a liquid crystal unit, which especially shows a long axis length of a liquid crystal layer; and

FIG. 11 is a schematic view of an X-ray imaging apparatus disclosed in U.S. Pat. No. 6,052,432.

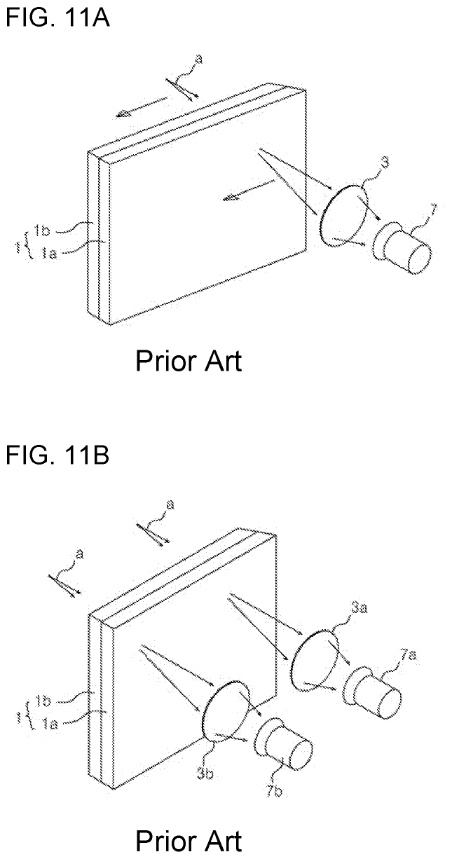

### DESCRIPTION OF THE PREFERRED EMBODIMENTS

The terminology used herein is for the purpose of describing particular embodiments only and is not intended to limit the scope of the present invention. As used herein, the singular forms “a”, “an”, and “the” are intended to include the plural forms as well unless the context clearly indicates otherwise. It will be further understood that the terms “comprises”, “includes”, or “has” when used in this specification specify the presence of stated features, regions, integers, steps, operations, elements and/or components, but do not preclude the presence or addition of one or more other features, regions, integers, steps, operations, elements, components and/or combinations thereof.

In addition, “on” or “above” means on or above an object, and does not necessarily mean an upper position based on the direction of gravity. Also, when a portion of a region, plate, or the like is referred to as being “on another portion or on top of another portion,” it may be directly on, be in contact with, spaced from the other portion, or another portion may be interposed between them.

It is also to be understood that when one element is referred to herein as being “connected to” or “coupled to” another element, it may be connected or coupled directly to the other element, or connected or coupled to the other element via a mediating element interposed therebetween, unless specifically stated otherwise.

In addition, terms used in the specification, “first”, “second”, etc., may be used to describe various components, but the components are not to be construed as being limited to the terms. These terms are used only for the purpose of distinguishing a component from another component.

Herein below, preferred embodiments, advantages, and features of the present invention will be described in detail with reference to the accompanying drawings.

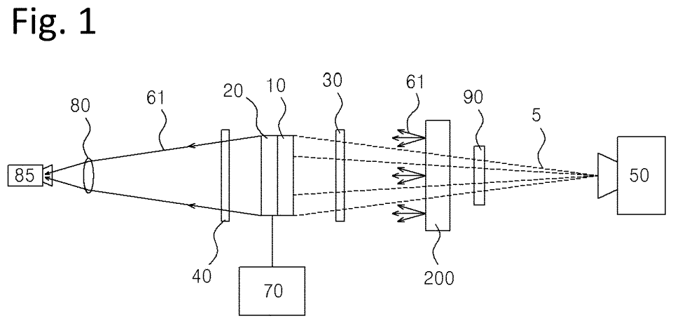

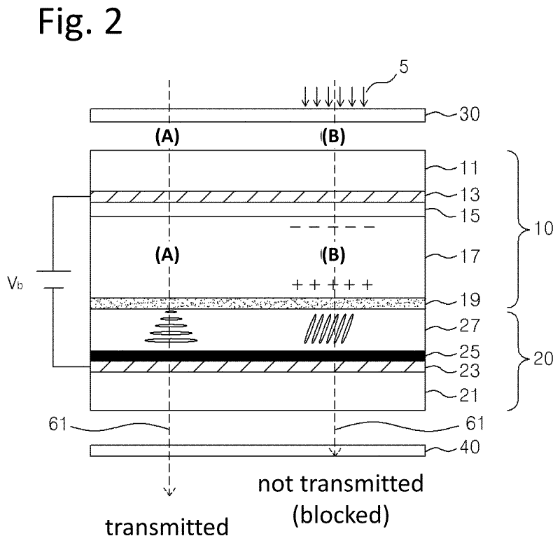

FIG. 1 is a diagram illustrating the overall construction of a liquid crystal X-ray detector according to one embodiment of the present invention, and FIG. 2 is a cross-sectional view of an X-ray sensing liquid crystal panel according to one embodiment of the present invention.

Referring to FIGS. 1 and 2, according to one embodiment of the present invention, a liquid crystal X-ray detector includes an X-ray output unit 50, an X-ray sensing liquid crystal panel 10 and 20, a read beam output unit 200, a driver unit 70, a polarizing plate 30, an analyzer 40, an imaging lens 80, and an image pickup unit 85.

The X-ray output unit 50 generates X-rays and outputs them to the outside. The X-rays output from the X-ray output unit 50 are transmitted through a subject 90 and then absorbed by a photoconductive layer 17 of the X-ray sensing liquid crystal panel.

The X-ray sensing liquid crystal panel includes a photoconductor unit 10 and a liquid crystal unit 20.

The photoconductor unit 10 of the X-ray sensing liquid crystal panel is configured such that a distribution of electrons and holes changes when it is irradiated with X-rays or applied with an electric field. Specifically, the photoconductor unit 10 includes a substrate 11, a transparent conductive film 13, an insulating film 15, a photoconductive layer 17, and an alignment film 19.

The substrate 11 (hereinafter, referred to as first substrate 11) of the photoconductor unit 10 is a base member on which the transparent conductive film 13, the insulating film 15, the photoconductive layer 17, and the alignment film 19 are to be formed. The substrate 10 is made of transparent glass or resin.

The transparent conductive film 13 (hereinafter referred to as “first transparent conductive film 13”) of the photoconductor unit 10 is an element to which a voltage is applied. The transparent conductive film 13 is formed on one surface of the first substrate 11 and is electrically connected to the driver unit 70 described below.

When a voltage is applied between the transparent conductive film of the photoconductor unit 10 and the transparent conductive film of the liquid crystal unit 20 by the driver unit 70 described below, a DC electric field is created. The electric field causes movement of electrons and holes in the photoconductive layer 17. That is, a distribution of electrons and holes changes in the photoconductive layer 17.

The first transparent conductive film 13 is made of a metal oxide such as indium tin oxide (ITO) or indium zinc oxide (IZO), or a metal oxide-metal-metal oxide such as indium tin oxide-silver-indium tin oxide (ITO-Ag-ITO).

The insulating film 15 of the photoconductor unit 10 is interposed between the first transparent conductive film 13 and the photoconductive layer 17 to prevent charge transfer between the first transparent conductive film 13 and the photoconductive layer 17.

The insulating film 15 is made of an insulating inorganic material such as silicon dioxide (SiO₂) or an insulating resin such as polycarbonate. The insulating film 15 is formed in the form of a thin film on one surface of the first transparent conductive film 13.

The photoconductive layer 17 of the photoconductor unit 10 is an element for creating charges. When the photoconductive layer 17 is irradiated with X-rays, a large number of electron-hole pairs are generated in the photoconductive layer 17. When this photoconductor layer 17 is exposed to an electric field, the electric field causes movement of electrons and holes. That is, a change in the distribution of charges occurs.

The photoconductive layer 17 is formed in the form of a thin film on the insulating film 15 and is made of selenium.

Preferably, the photoconductive layer 17 is made of amorphous selenium (a-Se). The amorphous selenium (a-Se) is formed through vacuum deposition or coating at low temperatures.

The alignment film 19 (hereinafter referred to as “first alignment film 19”) of the photoconductor unit 10 is an element that uniformly aligns the orientation of each of the liquid crystal molecules in conjunction with an alignment film 25 of the liquid crystal unit 20.

The first alignment film 19 can be formed through vacuum deposition of an inorganic material such as SiO₂ at a temperature below 40° C. Alternatively, the first alignment film 19 can be formed by polyimidizing polyamide, diluting the resulting polyimide with a low-temperature volatile solvent, applying the resulting diluted polyimide solution on the photoconductive layer 17 through wet coating, and firing the resulting polyimide coating formed on the photoconductive layer 17 for over one week in a vacuum furnace. Further alternatively, the first alignment film 19 can be formed by depositing parylene on the photoconductive layer 17 through vacuum deposition.

When the first alignment film is made of an inorganic material that has low anchoring energy and a low order parameter, the reliability of the liquid crystal is deteriorated. On the other hand, when the first alignment film is made of polyimide through low-temperature wet coating, there is a problem in that the residual solvent diffuses into the liquid crystal layer, thereby lowering the specific resistance of the liquid crystal and deteriorating the quality of an X-ray image. Therefore, it is preferable that the first alignment film 19 is formed through vacuum deposition of parylene.

When the first alignment film 19 is made of parylene, it is prepared through several processes including: a vaporization process of vaporizing parylene dimer; a decomposition process of decomposing the vaporized parylene dimer into parylene monomer by applying heat or plasma energy to the vaporized parylene dimer; a deposition process of depositing the parylene monomer on the photoconductive layer 17 at a temperature lower than 45° C. in a vacuum state to form a parylene layer; and a rubbing process of rubbing the parylene layer formed on the photoconductive layer 17.

More preferably, in the deposition process, it is preferable to maintain a process temperature below 40° C. that is 5° C. lower than the glass transition temperature Tg of amorphous selenium.

On the other hand, after the rubbing process is completed, a sealing member made of a thermosetting resin or a UV-curable resin is formed on the first alignment film 19 before the photoconductor unit 10 and the liquid crystal unit 20 are combined.

In the X-ray sensing liquid crystal panel in which the liquid crystal unit 20 and the photoconductor unit 10 are combined, the liquid crystal unit 20 functions to selectively transmit specific polarized wavelengths of a read beam. The liquid crystal unit 20 includes a substrate 21, a transparent conductive film 23, an alignment film 25, and a liquid crystal layer 27.

The substrate 21 (hereinafter, referred to as second substrate 21) of the liquid crystal unit 20 is a base member on which the transparent conductive film 23, the alignment film 25, and the liquid crystal layer 27 are to be formed. The second substrate 21 is made of transparent glass or polymer.

The transparent conductive film 23 (hereinafter referred to as “second transparent conductive film 23”) of the liquid crystal unit 20 is an element to which a voltage is applied. The second transparent conductive film 23 is formed on one surface of the second substrate 21 and is electrically connected to the driver unit 70 described below.

When a voltage is applied between the first transparent conductive film 13 and the second transparent conductive film 23, a DC electric field is generated between the first transparent conductive film 13 and the second transparent conductive films 23, thereby moving electrons and holes in the photoconductive layer 17. That is, a distribution of electrons and holes is changed in the photoconductive layer 17.

According to the preferred embodiment, the second transparent conductive film 23 is made of a metal oxide such as indium tin oxide (ITO) or indium zinc oxide (IZO), or a metal oxide-metal-metal oxide such as indium tin oxide-silver-indium tin oxide (ITO-Ag-ITO).

When the electric charge distribution in the photoconductor unit 10 changes due to X-ray irradiation and voltage application thereto, the orientation of the liquid crystals in the liquid crystal layer 27 of the liquid crystal unit 20 changes and accordingly the polarization transmission characteristic of the liquid crystal layer 27 with respect to the read beam changes. The liquid crystal layer 27 of the liquid crystal unit 20 includes a plurality of liquid crystal molecules injected into a gap between the first alignment film 19 and the second alignment film 25.

The alignment film 25 (hereinafter referred to as “second alignment film 25”) of the liquid crystal unit 20 is formed on the second transparent conductive film 23. When the liquid crystal unit 20 is bonded to the photoconductor unit 10, the second alignment film 25 is arranged to be symmetric to the first alignment film 19, thereby uniformly aligning the liquid crystal molecules in conjunction with the first alignment film 19.

Unlike the first alignment film 19, the second alignment film 25 can be formed without any constraint of temperature (for example, constraint of a temperature of 45° C. or higher). That is, the second alignment film 25 can be formed using the same process as in a conventional manufacturing method of a liquid crystal panel of a TN, STN, or TFT LCD device. The second alignment film 25 is formed by coating a surface of the cleaned second substrate 21 with a polyamide solution prepared by dissolving polyamide in a solvent, and then baking the polyamide coating at a temperature of about 150° C. for about 1 hour. Through this process, the polyamide is converted into polyimide. Thus, the second alignment film 25 made of polyimide can be formed. Here, the process of coating the surface of the second substrate 21 with polyamide may be performed through wet coating such as spin coating or printing.

Meanwhile, when the second alignment film 25 is formed with a polyamide solution to which spacers are added, a spacer scattering process can be omitted. That is, the second alignment film 25 to which spacers are fixed can be obtained by adding a small number of spacers to a polyamide solution which is a liquid crystal aligning agent, coating a substrate with the polyamide solution containing the spacers through spin coating or wet coating, and firing the resulting coating layer formed on the substrate.

After the second alignment film 25 is coated on the substrate, the second alignment film 25 is rubbed, and then a sealing member is formed on the rubbed second alignment film 25. The sealing member is made of a thermosetting resin or a UV-curable resin.

Thereafter, liquid crystals are scattered on the second alignment film 25. Thus, the liquid crystal unit 20 in which the second transparent conductive film 23, the second alignment film 25, and the liquid crystal layer 27 are sequentially formed on the second substrate 21 is obtained.

Next, the liquid crystal unit 20 is bonded to the photoconductor unit 10. Thus, the X-ray sensing liquid crystal panel according to the present invention can be manufactured through a one drop process.

In one embodiment of the present invention, the read beam output unit 200 is a device that emits a read beam 61 advancing toward the liquid crystal unit 20 of the X-ray sensing liquid crystal panel.

The read beam output unit 200 includes a light emitting unit that emits scattered light or plane light. The read beam 61 emitted from the read beam output unit 200 includes scattered light or plane light.

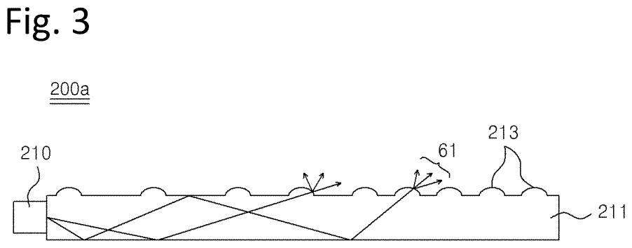

FIG. 3 is a cross-sectional view illustrating a read beam output unit according to a first embodiment of the present invention. Referring to FIG. 3, according to the first embodiment, a light emitting unit 200a of the read beam output unit 200 includes a light emitting element 210 and a light guide plate 211.

According to the first embodiment, the light emitting element 210 is a point light source such as a light emitting diode (LED), or is a tube-shaped line light source such as a cold cathode tube (fluorescent lamp or the like).

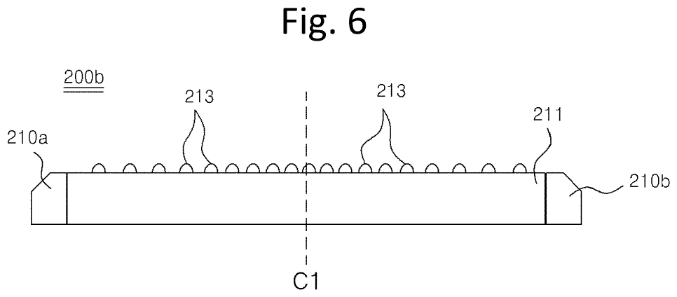

According to the first embodiment, the light emitting element is provided on one side surface of the light guide plate 211 as illustrated in FIG. 3, or the light emitting elements are provided on opposite side surfaces of the light guide plate 211, respectively as illustrated in FIG. 6. Here, the structure in which the light emitting element 210 is provided on one side surface of the light guide plate 211 refers to a structure in which the light emitting element 210 is disposed to face or to be in contact with a first side surface of the light guide plate 211.

When the light emitting element 210 is implemented with an LED, multiple light emitting elements may be arranged in an array. In this case, in the array of light emitting elements, the light emitting elements are arranged at intervals along the same axis.

In the first embodiment, the wavelength of the light emitted from the light emitting elements 210 range from 700 to 750 nm in a visible wavelength region. This will be described below.

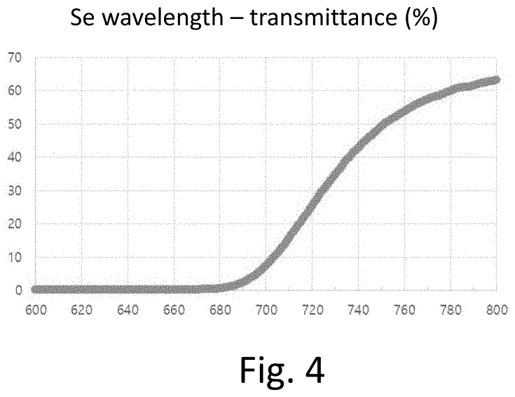

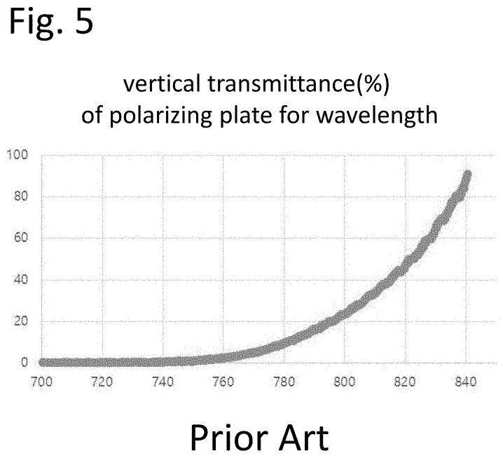

FIG. 4 is a light transmission curve of a photoconductive layer for each wavelength, according to one embodiment of the present. Referring to FIG. 4, when the photoconductive layer is made of amorphous selenium, the energy bandgap of the photoconductive layer is 2.2 eV. Therefore, light transmission through the photoconductive layer occurs from a wavelength of about 680 nm and is almost saturated at a wavelength near 800 nm. FIG. 5 is a view illustrating a per-wavelength vertical transmittance of a film type polarizing plate widely used in a conventional TFT LCD device. Referring to FIG. 5, it can be seen that light leakage begins to occur at a wavelength of 760 nm or longer and the performance of the polarizing plate is sharply deteriorated at a wavelength of about 800 nm. Therefore, when the photoconductive layer is formed of amorphous selenium, the light (i.e., read beam 61) output from the light emitting element needs to be a wavelength in a range of 680 to 760 nm. Preferably, the light needs to be a wavelength in a range of 700 to 750 nm.

In the first embodiment, the light guide plate 211 is an optical member that changes the optical distribution of the light beam focused on a narrow area such that the optical distribution of the light beam becomes uniform over a wide area. The light guide plate 211 totally reflects or refracts a point light beam or a line light beam incident thereon from the read beam output unit 200, thereby forming plane light with uniform luminance.

The light guide plate 211 is a thin plate or film made of a transparent resin material. For example, the light guide plate 211 may be made of an acrylic resin such as polymethylmethacrylate (PMMA). Alternatively, the light guide plate 211 may be made of a polycarbonate-based resin, a styrene-based resin, an olefin-based resin, a polyester-based resin, or the like.

The light guide plate 211 is provided with a light scattering pattern 213 on one surface thereof to provide a uniform plane light source. The light scattering pattern may be an embossed pattern composed of raised pattern elements having a hemispherical shape, a dot shape, a round-prism shape, a triangle-prism shape, or a lenticular shape. Alternatively, the light scattering pattern may be a recessed pattern composed recessed pattern elements having a U-cut shape, a V-cut shape, or a lenticular shape.

The light scattering pattern 213 is integrated with the light guide plate 211 for light scattering. The light scattering pattern 213 is formed through direct machining, etching, laser processing, or sandblasting.

The light scattering pattern 213 is composed of pattern elements that scatter light. The size of each of the pattern elements is smaller than the resolution of the liquid crystal X-ray detector. For example, in a case where the pattern element has a dot shape or a spherical shape and the resolution of the liquid crystal X-ray detector is 100 μm, the size (i.e., diameter) of the transverse long axis (i.e., diameter) of the pattern element of the light scattering pattern 213 on the light guide plate 211 is smaller than 100 μm.

When the light emitting element is provided only on one side surface of the light guide plate 211 as in the case of the light emitting unit 200a according to the embodiment (hereinafter, referred to as Embodiment 1a) illustrated in FIG. 3, the light scattering pattern 213 is configured in a manner that the spacing between each of the pattern elements is decreased as the distance from the light emitting element is increased. That is, the pattern elements are more densely arranged as the distance from the light emitting element is increased. This configuration of the light scattering pattern improves the brightness uniformity of the read beam emitted from the read beam output unit 200.

When the light emitting elements are provided on both side surfaces of the light guide plate 211, respectively as in the case of the light emitting unit 200b according to the embodiment (hereinafter, referred to as Embodiment 1b) illustrated in FIG. 6, the left light scattering pattern 213 on the left side of the center Cl of the light guide plate 213 is configured such that the spacing between each of the pattern elements adjacent to each other is gradually decreased toward the center Cl from the left light emitting element 210a. That is, the pattern elements of the light scattering pattern 213 in the left portion of the light guide plate 211 are most densely arranged at the center Cl of the light guide plate 211. In the light scattering pattern 213 on the right side of the center Cl of the light guide plate 211, the spacing between each of the pattern elements adjacent to each other is gradually decreased toward the center Cl of the light guide plate 211 from the right light emitting element 210b. That is, the pattern elements of the light scattering pattern 213 on the right side are most densely arranged at the center Cl of the light guide plate 211.

Alternatively, the light scattering pattern 213 may be configured such that the size of each pattern element (which is raised or recessed) is gradually increased as the distance to the center Cl of the light guide plate 211 is decreased.

According to the preferred embodiment, the read beam output unit 200 according to the first embodiment further includes a light incident portion 217 provided with a serration pattern 219.

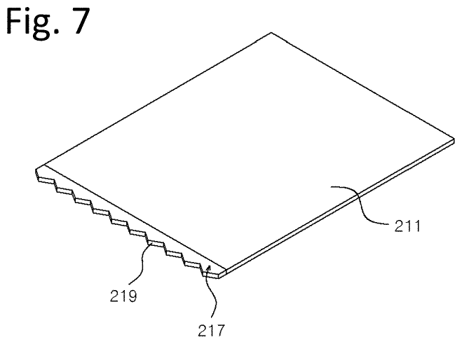

FIG. 7 is a perspective view of a light guide plate having a light incident portion, according to one embodiment of the present invention. Referring to FIG. 7, the light incident portion 217 is a region on which light emitted from the light emitting element is incident, and the light incident portion 217 is provided at a periphery portion of the light guide plate 211. The light incident portion 217 is integrally molded with the light guide plate 211. Alternatively, the light incident portion 217 is separately prepared from the light guide plate 211 and is then attached to one edge of the light guide plate 211.

The serration pattern 219 is formed on a light incident surface of the light incident portion 217. The light incident surface is a surface that receives the light emitted from the light emitting element. The serration pattern 219 functions to increase the angle at which light is incident and to broaden the scattering range of the incident light.

This will be described below. Although the light introduced into the light guide plate 211 refracts according to the refractive index of the light guide plate 211, dark areas in which the light beams do not overlap with each other may occur at the corners of the light guide plate 211. The term “periphery portion of the light guide plate 211” refers to an incident surface region on which the light emitted from the light emitting element is incident.

However, when the light guide plate 211 is provided with the serration pattern 219 on the light incident surface thereof, the light emitted from the light emitting element 210 refracts and diffuses at the serration pattern 219, thereby minimizing the dark areas. Therefore, the brightness uniformity of the plane light emitted from the read beam output unit 200 is improved.

The serration pattern 219 is composed of pattern elements having a V-cut shape or a U-cut shape. The pattern elements are arranged in the longitudinal direction of the light incident surface.

On the other hand, when the light emitting elements 210a and 210b are provided on both sides of the light guide plate as illustrated in FIG. 6 (Embodiment 1b), the light incident portion 217 includes a first light incident portion that is disposed in contact with or is arranged to face the first light emitting element 210a and a second light incident portion that is disposed in contact with or is arranged to face the second light emitting element 210b. The first and second light incident portions receive the light emitted from the first light emitting element 210a and the second light emitting element 210b, respectively. The light incident surface of each of the first and second light incident portions is provided with the serration pattern 219.

In the case of the read beam output units 200 according to Embodiment 1a and Embodiment 1b, the light emitted from the light emitting element 210 is introduced into the light guide plate 211, and the light is then totally reflected in the light guide plate 211 and is guided to reach even the corners of the light guide plate 211. However, since the light scattering pattern 213 is configured such that the light incident thereon is not totally reflected, the light can exit the light guide plate 211 and scatters in all directions. That is, plane light is emitted from the light guide plate 211.

Therefore, the read beam 61 is incident on the liquid crystal layer 27 as plane light. Therefore, even with the imaging lens 80 that is much smaller than the liquid crystal layer 27, a complete image of the subject 90 can be formed without requiring a task of combining partial images of the subject 90.

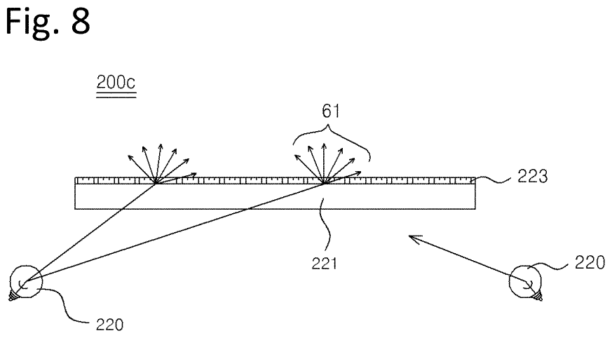

FIG. 8 is a cross-sectional view illustrating a read beam output unit according to a second embodiment of the present invention. Referring to FIG. 8, according to the second embodiment, a light emitting unit 200c of the read beam output unit 200 includes a light emitting element 220 and a light scattering plate 221.

According to the second embodiment, the light emitting element 220 is a point light source such as a light emitting diode (LED), or is a tube-shaped line light source such as a cold cathode tube (fluorescent lamp or the like).

The light emitting element 220 of the second embodiment is disposed such that light emitted from the light emitting element 220 is incident on the rear surface of the light scattering plate 221. Here, the rear surface of the light scattering plate 221 is the opposite surface to the front surface of the light scattering plate 221. The front surface of the light scattering plate 221 is a surface facing the polarizing plate. The front surface is also an emitting surface from which the read beam 61 is emitted.

As with the light emitting element 210 according to the first embodiment, the light (i.e., read beam 61) output from the light emitting element 220 needs to be light having a wavelength in a range of 680 to 760 nm and preferably light having a wavelength in a range of 700 to 750 nm.

According to the second embodiment, the light emitting element is a group of light emitting elements 220. In this case, the light emitting unit may include a first light emitting element that emits light in a first direction to the light scattering plate 221, a second light emitting element that emits light in a second direction different from the first direction, and an N-th light emitting element that emits light in an N-th direction different from each of the first and second directions.

According to the second embodiment, the light scattering plate 221 functions to scatter light incident thereon from the light emitting elements in all directions.

The light scattering plate 221 includes a substrate and a scattering surface 223. The substrate may be a glass substrate. The scattering surface 223 is provided with a light scattering pattern. In order to form the light scattering pattern including scattering pattern elements (i.e., embossed or recessed) having a size of 1 to 2 μm, a glass substrate is wrapped with a polishing material including 1 to 2 μm hydrocarbon particles. It is possible to improve the brightness uniformity of light by adjusting the structure of the embossed or recessed scattering pattern elements or the arrangement of the light emitting elements.

The light scattering pattern 213 is composed of multiple scattering pattern elements that scatter light. The size of each of the scattering pattern elements is finer than the resolution of the liquid crystal X-ray detector. For example, in a case where the scattering pattern elements are embosses and recesses and the resolution of the liquid crystal X-ray detector is 100 μm, the size of the transverse long axis of the scattering pattern element of the light scattering pattern 213 needs to be smaller than 100 μm.

According to the second embodiment described above, when the light emitted from the light emitting element 220 is incident on the light scattering plate 221, the light is scattered in all directions on the scattering surface 223 to reach the entire area of the front surface of the light scattering plate 221. In this case, the read beam 61 is incident as scattered light on the liquid crystal layer 27. Therefore, even with the imaging lens 80 that is much smaller than the liquid crystal layer 27, one complete image of a subject 90 can be obtained without requiring a task of combining multiple partial images of the subject.

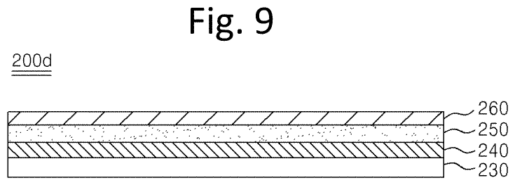

FIG. 9 is a cross-sectional view illustrating a read beam output unit according to a third embodiment of the present invention. Referring to FIG. 9, according to the third embodiment, a light emitting unit 200d of the read beam output unit 200 is implemented with an OLED plane light source.

Specifically, according to the third embodiment, the light emitting unit 200d includes a substrate 230 made of transparent glass or resin, an anode layer 240 formed on the substrate 230, an organic electroluminescent layer 250 formed on the anode layer 240, and a cathode layer 260 formed on the organic electroluminescent layer 250.

The anode layer 240 and the cathode layer 260 are connected to an external driver (not illustrated) via respective contact points (not illustrated) formed on the substrate 230. When an electric current is supplied to or a voltage is applied between the anode layer 240 and the cathode layer 230, the organic electroluminescent layer 250 emits plane light according to the applied voltage or current.

Alternatively, the OLED plane light source may be substituted with an inorganic EL light source in which an inorganic EL layer emits plane light.

As with the light emitting element in the first embodiment, the light (i.e., read beam) output from the OLED plane light source in the third embodiment needs to be light having a wavelength in a range of 680 to 760 nm and preferably light having a wavelength in a range of 700 to 750 nm.

According to the third embodiment described above, the read beam 61 emitted from the organic electroluminescent layer 250 is incident as plane light on the liquid crystal layer 27. Therefore, even with the use of an imaging lens 80 that is much smaller than the liquid crystal layer 27, a complete image of a subject 90 can be formed without requiring a task of combining partial images of the subject 90.

In the present invention, the driver 70 is a component that applies a predetermined bias voltage Vb between the first transparent conductive film 13 and the second transparent conductive film 23 to separate electrons and holes from electron-hole pairs.

In the present invention, the polarizing plate 30 is disposed on the light path between the photoconductor unit 10 and the read beam output unit 200, and the analyzer 40 is disposed on the light path in front of the liquid crystal layer 20, so that the transmittance of the read beam changes depending on the polarization transmittance characteristics of the liquid crystal layer 27.

In the present invention, the imaging lens 80 is disposed on the optical path in front of the analyzer 40. Therefore, the imaging lens 80 focuses the read beam transmitted through the analyzer so as to be imaged by an image pickup unit 85.

In a conventional liquid crystal X-ray detector, a read beam light source is implemented with a point light source. Therefore, light is emitted in a direction having a predetermined angle. Therefore, in order to translate the light exiting the liquid crystal layer into an image, an imaging lens that is larger than the liquid crystal layer is required.

However, since the liquid crystal X-ray detector according to the present invention is configured to emit scattered or plane light as the read beam 61, one complete image of a subject 90 can be formed without requiring a task of combining partial images of the respective parts of the subject 90.

Specifically, the long axis length of the imaging lens 80 is equal to or smaller than one half of the long axis length of the liquid crystal layer 27. The long axis length of the imaging lens 80 is preferably not longer than one-third (⅓), more preferably not longer than one-fifth (⅕), and most preferably not longer than one-tenth ( 1/10) of the long axis length of the liquid crystal layer 27. Even though the long axis length of the imaging lens 80 is equal to or smaller than one-tenth of the long axis length of the liquid crystal layer 27, an X-ray image having a suitable image quality and resolution for X-ray analysis of a subject can be obtained.

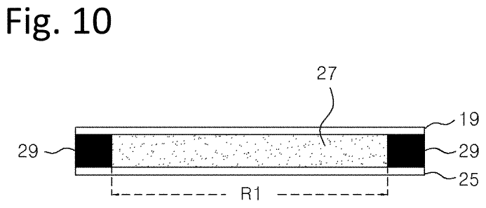

Here, the term “long axis length of the liquid crystal layer 27” refers to the length of the longest axis of a liquid crystal region distributed between the first alignment film 19 and the second alignment film 25. Referring to FIG. 10, the transverse length of the liquid crystal region sealed with the sealing member 29, i.e., the length denoted by reference numeral R1, corresponds to the long axis length of the liquid crystal layer 27. When the imaging lens 80 is spherical, the diameter of the imaging lens 80 corresponds to the long axis length of the imaging lens 80.

In the present invention, the image pickup unit 85 is a device that detects the read beam 61 delivered from the imaging lens 80 and produces an image from which a state of a subject can be diagnosed. The image pickup unit 85 is implemented with a CCD camera or a CMOS camera.

The distance between the image pickup unit 85 and the analyzer 40 varies depending on the viewing angle dependency of the liquid crystal. When the viewing angle dependency is small, the distance can be reduced.

The working principle of the liquid crystal X-ray detector will be described below.

As illustrated in FIG. 1, the liquid crystal X-ray detector according to one embodiment of the present invention has a structure in which the photoconductor unit 10 and the liquid crystal unit 20 are in face contact with each other. When the photoconductor unit 10 is exposed to X-rays, electrons and holes are created in the photoconductive layer 17. In this state, when a DC electric field is applied between the first transparent conductive film 13 and the second transparent conductive film 23, a polarization phenomenon occurs in which the electrons and the holes move to their opposite polarity side, i.e., to the first transparent conductive film and the second conductive film, respectively, for example, or vice versa.

Referring to FIG. 2, since a positive voltage is applied to the first transparent conductive film 13, the holes are distributed in a lower portion of the photoconductive layer 17 and the electrons are distributed in an upper portion of the photoconductive layer 17. That is, the holes gather in a region near the liquid crystal layer 27 and the electrons gather in a region near the first transparent conductive film 13.

This polarization phenomenon affects the liquid crystal layer 27, thereby changing the state of the liquid crystal. That is, when the charge distribution changes as shown in FIG. 2, the arrangement of liquid crystals in the liquid crystal layer 27 changes.

More specifically, the electrons and holes are separated in the photoconductive layer 17 irradiated with X-rays, thereby blocking the internal electric field of the photoconductive layer 17. Therefore, the voltage applied to the liquid crystal layer 27 increases.

In the example of FIG. 2, a voltage applied to liquid crystal cells in a region A which is not irradiated with X-rays differs from a voltage applied to liquid crystal cells in a region B which is irradiated with X-rays. Therefore, the liquid crystal layer 27 locally varies in the polarization transmittance characteristic with respect to the read beam depending on whether it is irradiated with X-rays or not. Because of the local variation in the polarization transmittance characteristics throughout the liquid crystal layer 27, the power of the read beam that exits the analyzer after passing through the polarizing plate 30 varies. Therefore, an X-ray image for diagnosing the state of a subject can be obtained from the light exiting the analyzer 40.

Referring to FIG. 4, the read beam incident on the region A can pass through the analyzer 40 but the read beam incident on the region B cannot pass through the analyzer 40.

Accordingly, the read beam that is emitted in the form of scattered light or plane light from the read beam output unit 200 sequentially passes through the polarizing plate 30, the photoconductor unit 10, the liquid crystal unit 20, and the analyzer 40 and is then selectively incident on the imaging lens 80. The image pickup unit 85 detects the light delivered from the imaging lens 80 to obtain an X-ray image of a subject.

Although preferred embodiments of the present invention have been described and illustrated using specific terms, it is apparent that those terms are used only for clarification of the present invention but not for limiting the scope of the present invention. Accordingly, it is apparent that those embodiments and terms can be modified, changed, altered, and substitutes without departing from the technical spirit and scope of the present invention as defined in the appended claims. It should be noted that modifications and equivalents to the embodiments fall within the scope of the present invention.

What is claimed is:

1. A liquid crystal X-ray detector comprising:
a photoconductor unit including a photoconductive layer; a liquid crystal unit provided on the photoconductor unit and including a liquid crystal layer; a read beam output unit configured to output a read beam toward the liquid crystal layer; and an imaging lens disposed on an optical path in front of the liquid crystal layer,
wherein the read beam output unit includes a light emitting unit configured to emit scattered light or plane light,
the read beam is scattering light or plane light, and
the imaging lens has a long axis length that is equal to or less than one-half of a long axis length of the liquid crystal layer.

2. The liquid crystal X-ray detector according to claim 1, wherein the long axis length of the imaging lens is equal to or less than one-tenth of the long axis length of the liquid crystal layer.

3. The liquid crystal X-ray detector according to claim 1, wherein the light emitting unit comprises a light emitting element and a light guide plate that converts light incident thereon from the light emitting element into plane light, and
the light emitting element is provided on at least one side surface of the light guide plate.

4. The liquid crystal X-ray detector according to claim 3, further comprising a light scattering pattern that is provided on the light guide plate and which is composed of a plurality of scattering pattern elements,
wherein each of the scattering pattern elements is smaller than a resolution of the liquid crystal X-ray detector.

5. The liquid crystal X-ray detector according to claim 3, further comprising a serration pattern composed of V-cut or U-cut pattern elements, the serration pattern being provided on an incidence surface of the light guide plate.

6. The liquid crystal X-ray detector according to claim 1, wherein the light emitting unit comprises a light emitting element and a light scattering plate that scatters the light incident thereon from the light emitting element in all directions, and
the light emitting element is disposed such that the light emitted from the light emitting element is incident on a rear surface of the light scattering plate.

7. The liquid crystal X-ray detector according to claim 6, wherein the light scattering plate has a light scattering pattern composed of a plurality of scattering pattern elements, and
each of the scattering pattern elements is smaller than a resolution of the liquid crystal X-ray detector.

8. The liquid crystal X-ray detector according to claim 6, wherein the light emitting element is a combination of a first light emitting element that emits light toward the light scattering plate in a first direction and a second light emitting element that emits light toward the light scattering plate in a second direction different from the first direction.

9. The liquid crystal X-ray detector according to claim 1, wherein the light emitting unit includes an organic electroluminescent diode or an inorganic electroluminescent diode.

10. The liquid crystal X-ray detector according to claim 1, wherein the photoconductive layer is formed of amorphous selenium, and
the read beam is light having wavelengths in a range of 680 to 760 nm.

11. The liquid crystal X-ray detector according to claim 1, further comprising:
an X-ray output unit that emits an X-ray;
a polarizing plate disposed on a light path in the rear of the photoconductor unit; an analyzer disposed on a light path in front of the liquid crystal display unit; and
an imaging unit that translates the read beam transmitted through the analyzer into an image.
